(19) United States
(12) Patent Application Publication
(10) Pub. No.: US 2026/0031181 A1
(43) Pub. Date: Jan. 29, 2026

(54) RIBONUCLEIC ACID TERTIARY STRUCTURE PREDICTION

(71) Applicant: UNIVERSITY OF MARYLAND, COLLEGE PARK, College Park, MD (US)

(72) Inventors: Pratyush TIWARY, College Park, MD (US); Lukas HERRON, Kensington, MD (US)

(21) Appl. No.: 19/278,449

(22) Filed: Jul. 23, 2025

Related U.S. Application Data
(60) Provisional application No. 63/674,623, filed on Jul. 23, 2024.

(51) Int. Cl. G16B 15/10; G16B 15/30; G16B 40/20

(52) U.S. Cl. CPC G16B 15/10; G16B 15/30; G16B 40/20

(57) ABSTRACT

A method may include obtaining a ribonucleic acid (RNA) sequence from a protein data bank comprising a plurality of RNA sequences. The method may also include generating a plurality of candidate RNA secondary structures by passing the RNA sequence through at least one RNA secondary structure prediction model. The method may further include assembling, via a RNA tertiary structure generator, the plurality of candidate RNA secondary structures into a plurality of RNA tertiary structures. The method may also include ranking the plurality of RNA tertiary structures by implementing a plurality of thermodynamic molecular machine learning models. The method may further include determining a structural binding affinity of small-molecule based on the ranked plurality of RNA tertiary structures.

### CROSS-REFERENCE TO RELATED APPLICATION

This application claims priority from U.S. provisional patent application No. 63/674,623 filed on Jul. 23, 2024. The contents of this earlier filed application are hereby incorporated by reference in their entirety.

### GOVERNMENT SUPPORT CLAUSE

This invention was made with government support under R35 GM142719 awarded by the National Institutes of Health and CHE2044165 awarded by the National Science Foundation. The government has certain rights in the invention.

### FIELD

Some embodiments may generally relate to protein structure prediction. Specifically, some embodiments may relate to systems and methods for ribonucleic acid (RNA) structure prediction. For example, certain embodiments may relate to apparatus, systems, and/or methods for predicting RNA tertiary structures.

### BACKGROUND

The majority of proteins in the human proteome—approximately 85%—are considered to be “undruggable”; their three-dimensional folds are incompatible with small molecule binding. Due to this, medicinal chemists have sought other avenues for targeting diseases caused by undruggable proteins, and in recent years, the potential of ribonucleic acids (RNA) as an alternative therapeutic target has been increasingly realized. Since the synthesis of proteins is regulated by RNA, small molecules targeting RNA structures can be used to indirectly control protein expression levels. However, much like for proteins, high-quality structural information is vital for the effective design of RNA-targeting drugs.

Despite recent advancements in the accuracy of protein structure prediction, the prediction of RNA tertiary structures remains a challenging problem. Advancements in protein structure prediction have been largely driven by the successful application of artificial intelligence (AI) to the structure prediction problem, where black-box models infer the relationship between sequence and structure from databases such as a Protein Data Bank (PDB). However, one cannot yet expect the same approach to work for RNA structure prediction since experimentally solved RNA tertiary structures are comparatively rare. The deposition rate of RNA structures in the PDB is two orders of magnitude lower than that of protein structures. This severe lack of data has presented a difficult-to-overcome bottleneck for the development of AI models for RNA tertiary structure prediction.

Experimental techniques for resolving biomolecular structures have become increasingly high-resolution, and as a result, there has been a paradigm shift in how biomolecules are best described. Advancements in structural biology have pointed away from the historical native-structure view in favor of a more disordered ensemble view. Within the ensemble view, it is not sufficient to predict a single conformation; multiple structures may be needed to account for the inherently dynamic nature of biomolecules. This is especially true for RNAs, which have more rotatable bonds per residue than proteins, rendering the one-structure-per-sequence paradigm even less accurate. As such, there is a need for RNA tertiary structure prediction methods to be able to generate a collection of possible competing structures that may exist at physiological temperatures, along with a thermodynamic ranking that accounts for energetic and entropic contributions to each structure's free energy.

### SUMMARY

Some embodiments may be directed to a method. The method may include obtaining a ribonucleic acid (RNA) sequence from a protein data bank comprising a plurality of RNA sequences. The method may also include generating a plurality of candidate RNA secondary structures by passing the RNA sequence through at least one RNA secondary structure prediction model. The method may further include assembling, via a RNA tertiary structure generator, the plurality of candidate RNA secondary structures into a plurality of RNA tertiary structures. In addition, the method may include ranking the plurality of RNA tertiary structures by implementing a plurality of thermodynamic molecular machine learning models. Further, the method may include determining a structural binding affinity of small-molecule based on the ranked plurality of RNA tertiary structures.

Other embodiments may be directed to an apparatus. The apparatus may include at least one processor and at least one memory including computer program code. The at least one memory and computer program code may be configured to, with the at least one processor, cause the apparatus at least to obtain a ribonucleic acid (RNA) sequence from a protein data bank comprising a plurality of RNA sequences. The apparatus may also be caused to generate a plurality of candidate RNA secondary structures by passing the RNA sequence through at least one RNA secondary structure prediction model. The apparatus may further be caused to assemble, via a RNA tertiary structure generator, the plurality of candidate RNA secondary structures into a plurality of RNA tertiary structures. In addition, the apparatus may be caused to rank the plurality of RNA tertiary structures by implementing a plurality of thermodynamic molecular machine learning models. Further, the apparatus may be caused to determine a structural binding affinity of small-molecule based on the ranked plurality of RNA tertiary structures.

Other embodiments may be directed to an apparatus. The apparatus may include means for obtaining a ribonucleic acid (RNA) sequence from a protein data bank comprising a plurality of RNA sequences. The apparatus may also include means for generating a plurality of candidate RNA secondary structures by passing the RNA sequence through at least one RNA secondary structure prediction model. The apparatus may further include means for assembling, via a RNA tertiary structure generator, the plurality of candidate RNA secondary structures into a plurality of RNA tertiary structures. In addition, the apparatus may include means for ranking the plurality of RNA tertiary structures by implementing a plurality of thermodynamic molecular machine learning models. Further, the apparatus may include means for determining a structural binding affinity of small-molecule based on the ranked plurality of RNA tertiary structures.

In accordance with other embodiments, a non-transitory computer readable medium may be encoded with instructions that may, when executed in hardware, perform a method. The method may include obtaining a ribonucleic acid (RNA) sequence from a protein data bank comprising a plurality of RNA sequences. The method may also include generating a plurality of candidate RNA secondary structures by passing the RNA sequence through at least one RNA secondary structure prediction model. The method may further include assembling, via a RNA tertiary structure generator, the plurality of candidate RNA secondary structures into a plurality of RNA tertiary structures. In addition, the method may include ranking the plurality of RNA tertiary structures by implementing a plurality of thermodynamic molecular machine learning models. Further, the method may include determining a structural binding affinity of small-molecule based on the ranked plurality of RNA tertiary structures.

Other embodiments may be directed to a computer program product that performs a method. The method may include obtaining a ribonucleic acid (RNA) sequence from a protein data bank comprising a plurality of RNA sequences. The method may also include generating a plurality of candidate RNA secondary structures by passing the RNA sequence through at least one RNA secondary structure prediction model. The method may further include assembling, via a RNA tertiary structure generator, the plurality of candidate RNA secondary structures into a plurality of RNA tertiary structures. In addition, the method may include ranking the plurality of RNA tertiary structures by implementing a plurality of thermodynamic molecular machine learning models. Further, the method may include determining a structural binding affinity of small-molecule based on the ranked plurality of RNA tertiary structures.

### BRIEF DESCRIPTION OF THE DRAWINGS

For proper understanding of example embodiments, reference should be made to the accompanying drawings, wherein:

FIG. 1A illustrates an example first stage RNA structure prediction pipeline, according to certain embodiments.

FIG. 1B illustrates an example second stage RNA structure prediction pipeline, according to certain embodiments.

FIG. 2A illustrates an example candidate model RNA structure refinement, according to certain embodiments.

FIG. 2B illustrates an example RNA structure arrangement across multiple refinements, according to certain embodiments

FIG. 3 illustrates an example of Boltzmann-scored ensembles of RNA structures, according to certain embodiments.

FIG. 4 illustrates an example flow diagram of a method, according to certain embodiments.

FIG. 5 illustrates an apparatus, according to certain embodiments.

### DETAILED DESCRIPTION

It will be readily understood that the components of certain example embodiments, as generally described and illustrated in the figures herein, may be arranged and designed in a wide variety of different configurations. The following is a detailed description of some example embodiments of systems, methods, apparatuses, and computer program products for predicting RNA tertiary structures.

The features, structures, or characteristics of example embodiments described throughout this specification may be combined in any suitable manner in one or more example embodiments. For example, the usage of the phrases “certain embodiments,” “an example embodiment,” “some embodiments,” or other similar language, throughout this specification refers to the fact that a particular feature, structure, or characteristic described in connection with an embodiment may be included in at least one embodiment. Thus, appearances of the phrases “in certain embodiments,” “an example embodiment,” “in some embodiments,” “in other embodiments,” or other similar language, throughout this specification do not necessarily refer to the same group of embodiments, and the described features, structures, or characteristics may be combined in any suitable manner in one or more example embodiments. As used herein, “models,” “structures,” or other similar language, throughout this specification may refer to RNA structures including, for example RNA tertiary structures, and the terms may be used interchangeably.

Additionally, if desired, the different functions or steps discussed below may be performed in a different order and/or concurrently with each other. Furthermore, if desired, one or more of the described functions or steps may be optional or may be combined. As such, the following description should be considered as merely illustrative of the principles and teachings of certain embodiments, and not in limitation thereof.

Certain embodiments provide for predicting thermodynamically ranked RNA tertiary structures combining deep learning, molecular simulations, and statistical mechanics. For instance, certain embodiments provide for predicting thermodynamics of RNA structure formation using generative artificial intelligence (GAI) capable of accelerating physics-based simulations via feedback with a GAI model. As such, certain embodiments may utilize GAI to obtain a ranked ensemble of all-atom RNA tertiary structures complete with solvent interactions that may be used for downstream applications such as, for example, in a drug delivery context.

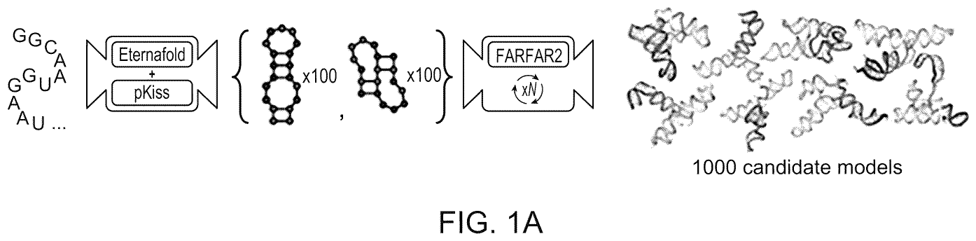

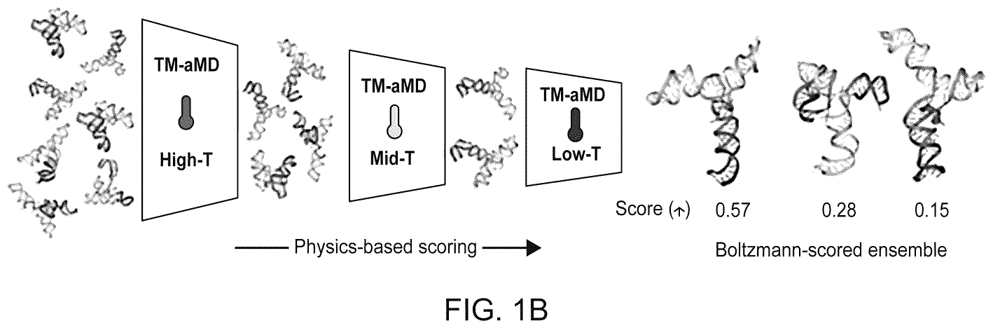

FIGS. 1A and 1B illustrate examples of a two-stage computational pipeline for Boltzmann-scored RNA ensembles, according to certain embodiments. The Boltzmann scores predict the temperature-dependent abundance of each conformer in the ensemble of RNA tertiary structures. As illustrated in FIG. 1A, in the first stage, a set of hypotheses (e.g., pool) of candidate RNA structural models are generated by taking into account bioinformatics and physics. According to certain embodiments, bioinformatics may include a prediction model such as, for example EternaFold (EF), and may include a Fragment Assembly with Full-Atom Refinement second (FARFAR2) model, employed as a method of modeling RNA tertiary structures.

According to certain embodiments, considering physics to generate the candidate RNA structural models may include the physics of base-pairing interactions (e.g., A-U and G-C) accounted for by pKiss. For a given sequence, pKiss solves an optimization problem to obtain the (approximate) minimum free energy secondary structure. FARFAR2 is a hybrid physics and bioinformatic algorithm that models RNA structures at coarse-grained and all-atom resolutions using energy functions with parameters from existing RNA structures. Additionally, FARFAR2 may be utilized to take a sequence and secondary structure as input, and return a tertiary model. FARFAR2 may produce the tertiary model by first guessing an initial structure, and then refining the initial structure through Monte Carlo sampling on the coarse-grained energy function. Once the refinement is completed, the all-atom details may be introduced and the structure may become relaxed in an all-atom potential.

Certain example embodiments molecular dynamics (MD) simulations model RNA structure at all-atom resolutions. The structures may be simulated using a forcefield (e.g., a set of energy parameters) that models bond angles, electrostatics, and solvent interactions. The MD may be carried out under a set of environmental conditions including, for example, temperature and pressure. In certain embodiments, the thermodynamic map (TM) algorithm introduces thermodynamics into the pipeline by modeling how the distribution of structures responds to changes in the environmental conditions (e.g., temperature). In some example embodiments, MD may be used to sample RNA tertiary structures displaying thermal fluctuations which are characteristic of structures at physiological temperatures. The MD may not be directly used to filter the tertiary models. Instead, the energy values and tertiary coordinates from the simulation may be used to train a thermodynamic map model, which may then be used to score and filter the tertiary structures. Filtering, in certain embodiments, may amount to keeping the conformers in, for example, the top 20% of scores, and discarding the structures that do not meet the score criteria.

In the second stage, the tertiary candidate structural models are scored by a thermodynamically-oriented AI model, and a representative Boltzmann-scored ensemble of RNA structures is selected from the candidate models.

RNA structures may be classified in ascending levels of detail including, for example primary structures, secondary structures, and tertiary structures. RNA primary structures may include the sequence of nucleobases, RNA secondary structures may include the base-pairings, and the tertiary structures may include the spatial positions of all atoms of the RNA structures. Following this hierarchy, the systems and methods for predicting thermodynamically ranked RNA tertiary structures may generate candidate models of the tertiary structures from secondary structures that are predicted from the primary RNA sequences (e.g., primary structure).

As illustrated in FIG. 1A, the first stage generates hypotheses (e.g., candidate RNA tertiary structures) by predicting RNA secondary structures. The number of possible secondary structures may grow exponentially with sequence length, and an exhaustive search for the most stable structure may become computationally intractable. However, according to certain embodiments, it may be possible to search for suboptimal secondary structures using simpler dynamic programming algorithms that reduce the search complexity from exponential to polynomial. A secondary structure may be classified as suboptimal when the structure is not the minimum free energy (MFE) secondary structure. The MFE secondary structure may correspond to a structure that minimizes the energy and maximizes the entropy, and may be the secondary structure of the RNA. In certain embodiments, EF and pKiss may predict the MFE structure, but both methods may also be able to sample other suboptimal structures. In certain embodiments, the closest match to the correct secondary structure may usually be a suboptimal structure. Thus, the methods and systems of certain embodiments may implement dynamic programming to form the basis of a wide variety of secondary structure prediction methods. According to certain embodiments, the prediction methods may include, but not limited to, for example, LinearFold, RNAstructure, Nussinov Algorithm, and ViennaRNA. Dynamic programming may rule out a class of secondary structures that contain long-ranged base pairs known as pseudoknots which may appear in RNA structures.

According to certain embodiments, dynamic programming may refer to a particular type of optimization algorithm that solves a difficult problem (in the sense of a long runtime and/or large search space) by recursively solving easier sub-problems. Solving the series of sub-problems may have a shorter runtime compared to the hard problem, and approximates the solution of the hard problem. In this example embodiment, the hard problem is finding the minimum free energy (MFE) secondary structure. The suboptimal structures are the structures that are obtained via solving the sub-problems.

As further illustrated in FIG. 1A, prior to generating the candidate models, the RNA sequences may serve as input into RNA secondary structure prediction models such as, for example EF and pKiss (PK) to generate RNA secondary structures. EF may differ from pKiss which is physics-based, where a key difference is that the bioinformatic algorithm relies on large amounts of data to train a model with many free parameters to predict secondary structures. In contrast, the physics-based algorithm (pKiss) predicts structures by using a small set of energy parameters and solving an optimization problem. Similarly, the FARFAR2 model makes use of an extensive library of RNA structure motifs (e.g., “fragments”) to accelerate sampling of RNA structures. According to certain embodiments, EF may correspond to an algorithm for predicting pseudoknot-free RNA structures, while PK may correspond to one of multiple algorithms capable of predicting diverse classes of pseudoknots. In the pipeline of FIG. 1A, the EF and PK may generate RNA secondary structures that can later be assembled and refined into models of the tertiary structures.

According to certain embodiments, the number of RNA secondary structures generated may include thousands of potential structures. For example, pKiss may produce hundreds of secondary structures. Although FIG. 1A illustrates that hundreds of secondary structures are generated, the actual number of secondary structures obtained may depend on the length of the RNA (e.g., longer RNAs have more potential secondary structures).

As illustrated in FIG. 1A, the RNA tertiary structures (e.g., 1,000 candidate models) may be assembled by inputting the generated RNA secondary structures into a FARFAR2 model. The FARFAR2 model may be configured to stitch together three-residue fragments of RNA secondary structures whose sequences match the target sequence. For example the FARFAR2 model may use Monte Carlo simulations to assemble an extensive library of the RNA structural motifs into a tertiary structure, with the ability to condition the assembly on a secondary structure. According to certain embodiments, the assembly may be conditioned by providing a secondary structure as input (e.g., extra information) to the FARFAR2 model. The FARFAR2 model may be able to generate tertiary models without any secondary structure information (e.g., with just the sequence as input), however, certain embodiments may be provided with secondary structures. The Monte Carlo process may be guided by a low-resolution scoring function that rewards base pairs and base stacks with geometries similar to those seen in the generated RNA secondary structures. The low-resolution scoring may correspond to a function that is a sum of six terms that encode the most relevant aspects of the RNA structure. The terms may include: (1) a term that favors overall compactness of the molecule; (2) a term that penalizes clashes (non-physical overlap) between atoms; (3) a term that favors base-pairing; (4, 5) two terms that encourage co-planarity of paired bases; and (6) a term that encourages base stacking. Each candidate RNA tertiary structure may be refined in a high-resolution all-atom scoring function that rewards hydrogen bonds, van der Waals packing of atoms, and other physically important interactions, and the lowest-energy models may be clustered to achieve submitted models. In some embodiments, the FARFAR2 model may implement a special set of Monte Carlo moves for nucleotides in stacked Watson-Crick pairs (e.g., base-pair steps) that maintain Watson-Crick geometry of RNA helices while allowing their backbone conformations to be perturbed. The high-resolution structure model may include interactions at the atomic level. For example, each base-pair interaction may be a single term in the low-resolution potential, but in the high-resolution potential, each base-pair may be modeled by pairwise interactions between all involved atoms. The difference between high- and low-resolution is in the number of terms (e.g., 6 for the low-resolution potential vs many more for the all-atom score function). Additionally, the FARFAR2 model may implement one or more score filters and chainbreak filters during fragment assembly to allow recognition of poorly assembled RNA tertiary structure conformations that can be discarded before the all-atom minimization, leading to more efficient use of the computational power of the FARFAR2 model. For instance, in some embodiments, the chainbreak filter may specify that the RNA tertiary structure should not include chainbreaks worse than a certain unit length (e.g., 12.0 Å). In some embodiments, the score filter may specify that the RNA tertiary structure must be in the top quintile of low-resolution scores. In certain example embodiments, in FIG. 1A, the tertiary models may be filtered based on the score assigned by FARFAR2, while in FIG. 1B, the tertiary models may be filtered based on the score (e.g., likelihood) assigned by the TM model.

According to certain embodiments, an energy function for the assembly of the RNA tertiary structures may be low-resolution potential, with terms and weights derived from biophysical principles and experimental data. The energy function may correspond to the score function. For instance, when the energy or score function is low-resolution, it may be referred to as a score function. The assembly algorithm (e.g., FARFAR2) may optimize the score/energy. The score/energy function may be configured such that known, solved RNA structures lie at the minimum. Additionally, the best models from the low-resolution potential may be refined in a higher-resolution potential yielding more realistic structures. According to certain example embodiments, the refinement may refer to the fact that the tertiary structures are subject to changes over the course of each simulation so that the energy is minimized.

In certain embodiments, the resolution of the RNA tertiary structures may be increased (e.g., the accuracy of the RNA tertiary structure may be improved through refinement in successively high-resolution energy functions) by iteratively refining the FARFAR2-generated models in a high-resolution MD forcefield. According to certain embodiments, the resolution may correspond to the types and number of interactions modeled by the score functions. The FARFAR2 low-resolution function may include six terms and have a smooth structure, making it ideal for sampling. The high-resolution FARFAR2 score function may include more terms and may be suitable for energy minimization of the structures produced by the low-resolution function. In some embodiments, the forcefield may represent the most accurate energy function. The forcefield may include the most terms and model the greatest variety of interactions.

In certain embodiments, the forcefield may represent an energy function similar to those employed by FARFAR2. A difference between the forcefield and the energy function employed by FARFAR2 is that the forcefield includes many more terms (and hence more physical details) that account for electrostatics, solvent, and bonded and non-bonded interactions. Additionally, all of the forcefield terms may be meant to model the interactions between pairs of atoms. Another difference between the Rosetta score functions and the forcefield is that the forcefield is rougher compared to the Rosetta functions due to the additional terms. As such, the forcefield may be less effective for generating tertiary models compared to FARFAR2 models. The purpose of using the forcefield may be to model how the RNA structure behaves under thermal fluctuations.

RNA tertiary structures with high energy under the MD forcefield may be discarded, and additional FARFAR2 models (e.g., RNA tertiary structures) may be generated from the RNA secondary structures of the remaining models. According to certain embodiments, the iterative procedure may steer the FARFAR2 ensemble towards regions of low energy in the MD forcefield. Additionally, the sequential prediction of the RNA secondary and RNA tertiary structures may yield thousands of candidate hypotheses for physics-based scoring, as illustrated in FIG. 1A.

As illustrated in FIG. 1B, a small number of models (e.g., an ensemble) are selected from the group of candidate RNA tertiary structures obtained from implementing the FARFAR2 model. The ensemble at the second stage of the pipeline represents the diversity of RNA structures at different physiological temperatures. The prediction model/method of certain embodiments may select representative RNA tertiary structures by simulating the RNA tertiary structures obtained in the first stage of the pipeline illustrated in FIG. 1A. For example, in some embodiments, the simulation may be performed by applying molecular dynamics. According to certain embodiments, simulating may refer to the molecular dynamics simulations. Representative structures maybe selected and simulated for 100 nanoseconds using a RNA forcefield and an implicit solvent model. In one example embodiment, an OpenMM simulation engine may be used. The molecular dynamics simulations may be where temperature and ions are introduced, which are not considered by the FARFAR2 model. The simulated RNA structure may exhibit thermal fluctuations, which can be essential for training the TM.

According to certain embodiments, application of MD may require a starting structure which is passed as input to a simulation engine (e.g., OpenMM) along with various simulation parameters (e.g., duration, timestamp, temperature, pressure, etc.) and the forcefield files. In some embodiments, the MD simulations may not be directly used for selecting the RNA conformers. The TM model may be trained on coordinates and forcefield energy values may be extracted from simulations. The TM may then predict a score which may be used to filter the models. Additionally, the Rosetta score (e.g., FARFAR2's high-resolution energy function) may be used to select the representative structures that can later be simulated via MD.

In some embodiments, a thermodynamic map (TM) may actively steer and score the simulations. The “steer” may refer to TM-aMD protocol described herein, and may refer to simulating the structures for 100 ns each, train a TM model, use that model to score the structures, and then select new representatives based on the score predicted by the TM model. Another round of simulations may be performed from these new representatives, and another TM may be trained to estimate the scores. These iterative procedures may be referred to as “steering.”

According to certain embodiments, the score may represent the probability of a given structure, at a given temperature, as estimated by the TM. In other instances, such as, for example, diffusion models, the score may be referred to as a “likelihood estimation.” In other embodiments, the TM may be trained on all of the RNA conformations produced by this round of MD simulation. The TM may incorporate score-based generative modeling into the framework of free energy perturbation within statistical mechanics. Additionally, the TM maps the temperature dependence of ensembles of configurations of a complex system onto the temperature dependence of a simple, idealized system which allows for efficient generation of physically realistic samples of the complex system with the correct Boltzmann weights (e.g., scores).

According to certain embodiments, the TM may represent a generative AI model that learns the equilibrium distribution of structures and the effect of temperature on the equilibrium. In certain embodiments, the structures of the equilibrium distribution may correspond to the RNA tertiary structures. Additionally, the equilibrium distribution may represent a distribution of structures subject to some environmental constraints including, for example, temperature. In some embodiments, the distribution of RNA structures (e.g., the probability of a given structure) may change in response to environmental factors such as, for example, temperature. The TM learns the equilibrium distribution of the structures by parameterizing an invertible mapping between finite-temperature structures and a generative system (e.g., a harmonic oscillator) at the same temperature. The finite-temperature structures may refer to the fact that the structures are generated by MD simulations at a non-zero temperature, and are therefore representative of fluctuating RNA structures. Methods such as FARFAR2 may not model the thermal fluctuations and, thus, their tertiary structures may be representative of zero-temperature RNA structures. The TM may take the form of a forward diffusion process that maps samples onto the generative system and the forward process' inverse. In some embodiments, the forward diffusion may be constructed to have a closed-form solution, from which a score-based model parameterizes the inverse. Once parameterized, samples (e.g., RNA tertiary structures represented as feature vectors) may be generated at any temperature T by first sampling states of the generative system at T and evaluating the inverse map. The effect of temperature on the ensemble may be implicitly accounted for in the construction of the mapping. According to certain embodiments, the all-atom coordinates may be used to compute a distance map between the bases, and then perform dimensionality reduction through Principal Component Analysis (PCA) or a State Predictive Information Bottleneck (SPIB) model. The end result may be a set of vectors that represent the all-atom conformers.

In certain embodiments, the framework of TMs infer the temperature dependence of the distribution of RNA tertiary structures, stability of specific conformations of the RNA tertiary structures, and macroscopic thermodynamic properties of the RNA tertiary structures such as melting curves and heat capacities. According to certain embodiments, a system (e.g., the entities that are simulated via MD including, for example, ions in addition to the RNA tertiary structures) may be driven towards equilibrium. The system of certain embodiments may be driven towards equilibrium by introducing feedback between a thermodynamic map and molecular dynamics simulations. For example, in some embodiments, the temperature dependence of the generative system's equilibrium may be used to actively steer the simulations (from which the thermodynamic map is learning) until convergence is reached (e.g., when the distribution of structures stops changing from iteration to iteration). Once converged, the free energy estimated by the thermodynamic map may be used as the Boltzmann score.

According to certain embodiments, the hypothesis generation illustrated in the first stage of the pipeline in FIG. 1A may be performed at the secondary structure level. After performing the hypothesis generation, a feedback (e.g., iterative simulate-train-score-simulate approach, where the model determines representative structures for the next round of simulation) may be implemented in a similar form to thermodynamic map-accelerated molecular dynamics (TM-aMD) protocol, wherein simulations are adaptively re-seeded based on the generated equilibrium distribution. According to some embodiments, a simulated annealing procedure may be implemented to stabilize training and facilitate scoring the RNA tertiary structures.

According to certain example embodiments, the RNA tertiary structure prediction model may be back-tested on three RNAs by comparing the root mean square deviation (RMSD) of the RNA tertiary structure prediction model against other methods. Details of the tested RNAs are shown in Table 1.

TABLE 1
Summary of backtesting results

| | | | Best RNA | RNA |
|---|---|---|---|---|
| | | | tertiary | tertiary |
| | | Best | prediction | prediction |
| | | Candidate | model | model |
| | Length | RMSD | RMSD | placement |
| Name (Code) | (nt) | (Å) | (Å) | (Top %) |
| CPEB3 ribozyme (1107) | 69 | 6.82 | 6.51 | 9.2 |
| Cloverleaf RNA (1116) | 157 | 15.3 | 15.3 | 22 |
| SARS-CoV-2-SL5 (1149) | 124 | 11.3 | 11.3 | 7.2 |

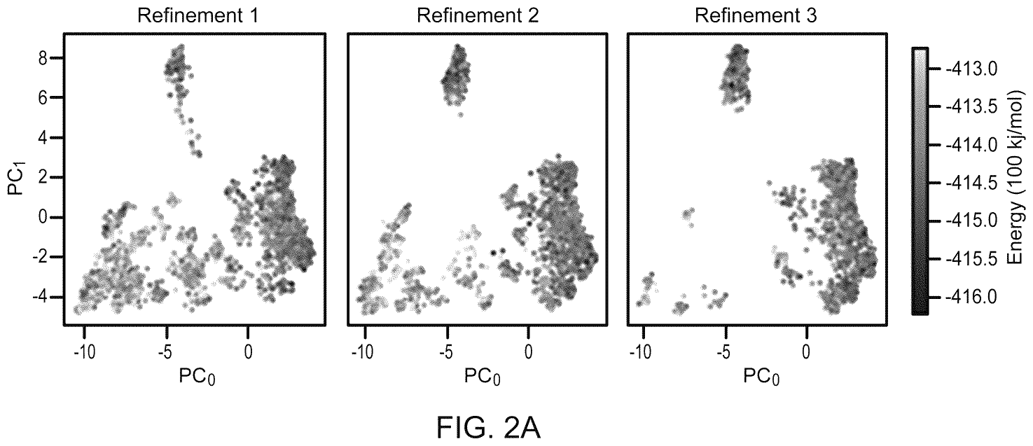

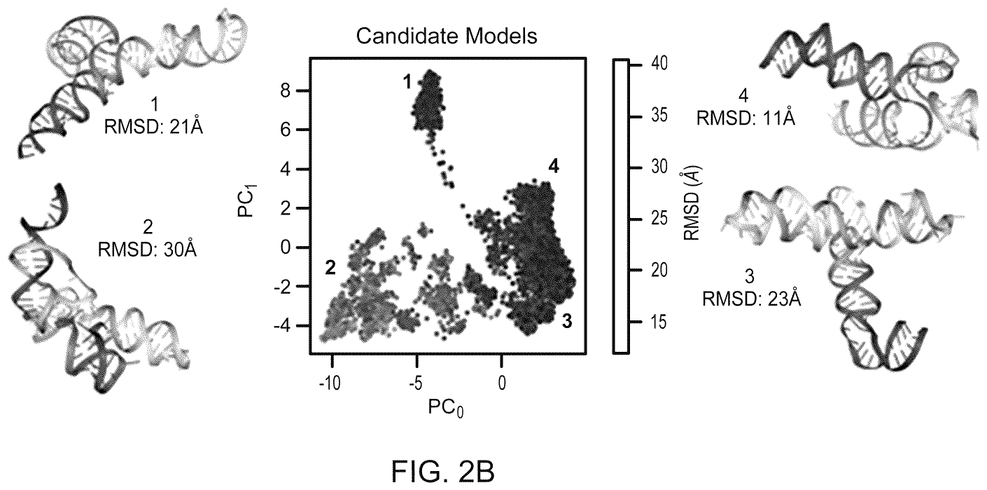

According to certain embodiments, the performance of each stage of the pipeline illustrated in FIGS. 1A and 1B may be evaluated independently because the candidate models' accuracy limits the final ensemble's accuracy. FIG. 2A illustrates a distribution of candidate models for each refinement projected along two principal components, according to certain embodiments. The models are distinguished by their color-shaded distribution, wherein the shading correspond to the models' energy in the MD forcefield, which demonstrates how the FARFAR2 assembly is steered towards regions of low energy. In certain embodiments, samples of RNA tertiary structures may be aggregated across refinements to form a set of candidate models.

FIG. 2B illustrates the candidate models where the shaded coloring is a representation of the candidate models RMSD from the native structure, with the closest model to the native structure having an RMSD of 11A. The low energy clusters sampled by the steered FARFAR2 assembly process correspond to the lowest RMSD models. As illustrated in FIG. 2B, multiple clusters in the projection suggest structural diversity among the candidate models (e.g., RNA tertiary structures), and representative structures are illustrated from four of the clusters. Table 1 shows the RMSD of the best candidate model generated by the RNA tertiary structure prediction model. Table 1 also shows that, for each of the RNAs tested, the scoring stage of the RNA tertiary structure prediction model of certain embodiments selects the optimal hypothesis.

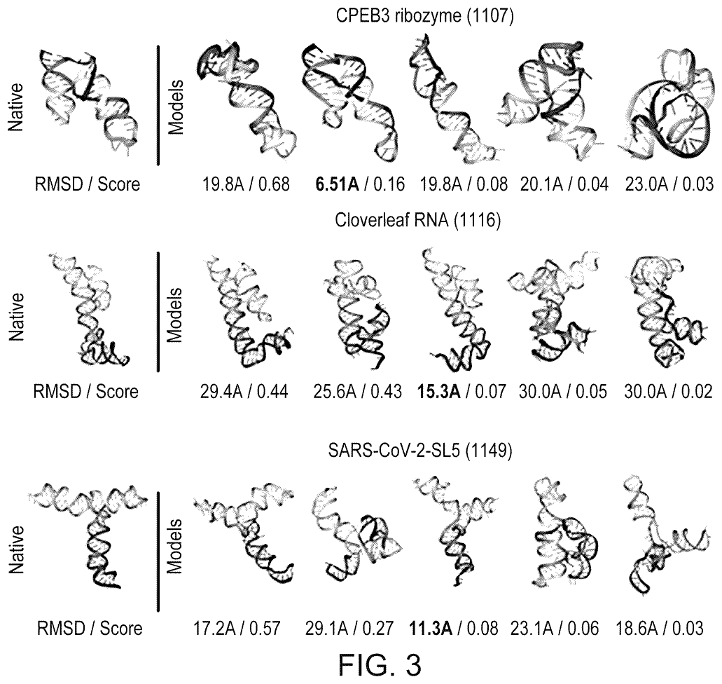

In certain embodiments, the top five models selected by the RNA tertiary structure prediction model for each RNA are illustrated in FIG. 3. Specifically, FIG. 3 illustrates Boltzmann-scored ensembles for blind structure prediction competition systems (the system referring to a particular RNA sequence whose structure is to be predicted), according to certain embodiments. The participants of the blind structure prediction competition know only the sequence of the target and nothing about the structure. As illustrated in FIG. 3, the Boltzmann-scored ensembles are depicted for 1107, 1116, and 1149 RNAs. The RMSDs of the models of the ensembles may be determined between the native structure (left) and the models obtained from the RNA tertiary structure prediction model of certain embodiments (right). The RMSD and Boltzmann scores are shown for each model, with the closest model's RMSD as 6.51 Å for RNA 1107, 15.3 Å for RNA 1116, and 11.3 Å for RNA 1149. The contents of FIG. 3 indicate that the algorithm for generating candidate models in FIG. 1A can predict the global structure of the fold. In other embodiments of the RNA tertiary structure prediction model, delicate structural elements such as for example, junctions and turns containing non-canonical (non-Watson-Crick) interaction, may be modeled using an energy function in the FARFAR2 assembly or through homology and template modeling.

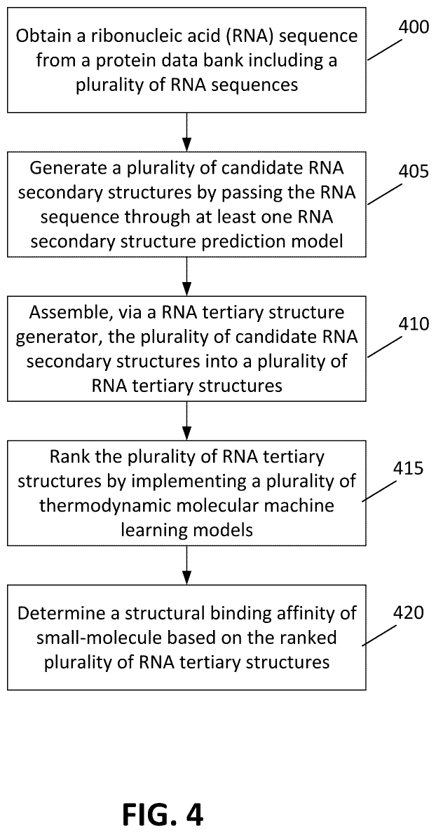

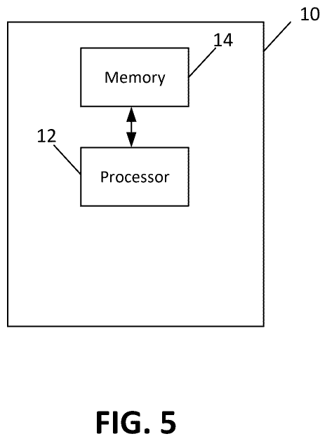

FIG. 4 illustrates an example flow diagram of a method, according to certain example embodiments. In certain example embodiments, the flow diagram of FIG. 4 may be performed by a system that includes a computer apparatus, computer system, network, neural network, apparatus, communication device, mobile computer, mobile communication device, or other similar device(s). According to certain embodiments, each of these apparatuses of the system may be represented by, for example, an apparatus similar to apparatus 10 illustrated in FIG. 5.

According to one example embodiment, the method of FIG. 4 may include, at 400, obtaining an RNA sequence from a protein data bank comprising a plurality of RNA sequences. The method may also include, at 405, generating a plurality of candidate RNA secondary structures by passing the RNA sequence through at least one RNA secondary structure prediction model. The method may further include, at 410, assembling, via a RNA tertiary structure generator, the plurality of candidate RNA secondary structures into a plurality of RNA tertiary structures. Further, the method may include, at 415, ranking the plurality of RNA tertiary structures by implementing a plurality of thermodynamic molecular machine learning models. In addition, the method may include, at 420, determining a structural binding affinity of small-molecule based on the ranked plurality of RNA tertiary structures.

According to certain embodiments, the method may also include filtering the plurality of candidate RNA tertiary structures based on energetic characteristics of the plurality of RNA tertiary structures assembled via the RNA tertiary structure generator. According to some example embodiments, the method may further include filtering the RNA tertiary structures obtained through a molecular dynamics simulation based on a score predicted by a thermodynamic map model. According to other embodiments, the energetic characteristics may include at least one of hydrophobicity, electrostatics, or ion addition. In certain example embodiments, MD may be used to sample RNA tertiary structures displaying thermal fluctuations which are characteristic of structures at physiological temperatures. Although the MD may not be directly used to filter the tertiary models, the energy values and tertiary coordinates from the simulation may be used to train a thermodynamic map model, which may then be used to score and filter the tertiary structures. According to certain embodiments, the filtering may amount to keeping the conformers in the, for example, top 20% (or other percentage) of scores and discarding the rest.

In certain embodiments, the plurality of RNA tertiary structures are assembled based on implementation of Monte Carlo simulations to condition the plurality of RNA tertiary structures. In certain embodiments, the conditioning of the RNA tertiary structures may include passing a secondary structure as input to FARFAR2 in addition to the sequence, as opposed to passing only the sequence. In some embodiments, the method may also include refining the plurality of RNA tertiary structures through simulation under a series of increasingly high-resolution energy functions, each of which models physical interactions in greater detail compared to those prior. In certain embodiments, the energy function functions may refer to low-resolution FARFAR2 energy function, the high-resolution FARFAR2 energy function, and the MD forcefield (also an energy f unction). In some embodiments, an increasing number of physical details (e.g., terms in the energy function) may be added as one progresses from simulating the RNA tertiary structure using low-resolution FARFAR2, high-resolution FARFAR2, and then MD. In other embodiments, the refinement may relate to the tertiary structures undergoing changes over the course of each simulation so that the energy can be minimized.

According to certain embodiments, the method may further include simulating the plurality of candidate RNA secondary structures with molecular dynamics. According to some embodiments, the simulation may correspond to that illustrated in FIG. 1B. According to certain embodiments, the simulation may refine the tertiary structure and sample RNA structures that exhibit variations in structure due to thermal fluctuations. Simulating may include evolving the RNA structure in a time-dependent manner using the energy function. For MD simulations, Newton's equations of motion may be iteratively solved. There may be multiple ways of simulating dynamics of an RNA, and MD may represent one method. For example, FARFAR2 may use a Monte Carlo procedure which may be fundamentally different from MD.

In certain embodiments, the method may also include training models based on features of the plurality of RNA tertiary structures, and extracting a score from the trained models. According to certain embodiments, the model may be a neural network, which may be a useful element of the thermodynamic map algorithm. The model may be trained on RNA structures. For instance, all-atom RNA coordinates and energy values may be extracted from the MD simulations. Then, the all-atom coordinates may be represented as feature vectors. The neural network may then be trained on the feature vectors and energy values. According to some embodiments, the score may correspond to a quantity derived from the neural network. For instance, for a given feature vector (corresponding to an RNA structure), the probability or likelihood of the vector may be estimated from the neural network. This likelihood may correspond to the score, and the score may be used to rank conformers by, for example, sorting the conformers from high likelihood to low likelihood. The conformers with the highest likelihood may be considered as the highest ranked.

According to certain embodiments, the ranking of the plurality of RNA tertiary structures may be based on temperature and entropy, and external environmental conditions of the RNA sequence. According to some embodiments, the temperature may correspond to the temperature that the MD simulations are conducted at. The value may be determined by the user based on its particular use case. For instance, one example may be human physiological temperature of 98.7° F. According to certain embodiments, entropy may refer to the entropy of the RNA tertiary structure. For a given temperature, there may be many compatible tertiary structures. The entropy may quantify the number of compatible structures. In some embodiments, the thermodynamic map models may learn the entropy of the RNA structure as a function of temperature. The temperature and entropy may change the ranking. At a cold temperature (e.g., low entropy), well-folded structures may have a high likelihood as predicted by the model, but well-folded structures may have a low likelihood. Instead, unfolded structures may be ranked highly by the model. In some embodiments, the temperature may affect the ranking because RNA structures fold and melt in response to changes in temperature.

In certain embodiments, the method may also include generating a thermodynamic map of the plurality of RNA tertiary structures based on an equilibrium distribution of the plurality of RNA tertiary structures, and the effect of temperature on the equilibrium distribution. In some embodiments, the equilibrium distribution may represent a probability distribution defined over the RNA tertiary structures. The equilibrium distribution may have a particular mathematical form, and may model the probability of observing a tertiary structure subject to certain environmental constraints such as, for example, temperature and/or pressure.

FIG. 5 illustrates an apparatus 10 according to an example embodiment. In certain embodiments, although only one apparatus 10 is illustrated, apparatus 10 may be apparatus representing multiple apparatuses as part of a system or network. For example, in certain embodiments, apparatus 10 may be a computer or communication device, mobile computer or communication device, or computer apparatus that operates individually or together in a computer system or computer network system.

In some embodiments, the functionality of any of the methods, processes, algorithms or flow charts described herein may be implemented by software and/or computer program code or portions of code stored in memory or other computer readable or tangible media, and executed by a processor.

For example, in some embodiments, apparatus 10 may include one or more processors, one or more computer-readable storage medium (for example, memory, storage, or the like), one or more radio access components (for example, a modem, a transceiver, or the like), and/or a user interface. It should be noted that one of ordinary skill in the art would understand that apparatus 10 may include components or features not shown in FIG. 9.

As illustrated in the example of FIG. 5, apparatus 10 may include or be coupled to a processor 12 for processing information and executing instructions or operations. Processor 12 may be any type of general or specific purpose processor. In fact, processor 12 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. While a single processor 12 is shown in FIG. 5, multiple processors may be utilized according to other embodiments. For example, it should be understood that, in certain example embodiments, apparatus 10 may include two or more processors that may form a multiprocessor system (e.g., in this case processor 12 may represent a multiprocessor) that may support multiprocessing. According to certain example embodiments, the multiprocessor system may be tightly coupled or loosely coupled (e.g., to form a computer cluster).

Processor 12 may perform functions associated with the operation of apparatus 10 including, as some examples, precoding of antenna gain/phase parameters, encoding and decoding of individual bits forming a communication message, formatting of information, and overall control of the apparatus 10, including processes illustrated in FIGS. 1-4.

Apparatus 10 may further include or be coupled to a memory 14 (internal or external), which may be coupled to processor 12, for storing information and instructions that may be executed by processor 12. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and/or removable memory. For example, memory 14 can be comprised of any combination of random access memory (RAM), read only memory (ROM), static storage such as a magnetic or optical disk, hard disk drive (HDD), or any other type of non-transitory machine or computer readable media. The instructions stored in memory 14 may include program instructions or computer program code that, when executed by processor 12, enable the apparatus 10 to perform any of the various tasks described herein.

In certain embodiments, apparatus 10 may further include or be coupled to (internal or external) a drive or port that is configured to accept and read an external computer readable storage medium, such as an optical disc, USB drive, flash drive, or any other storage medium. For example, the external computer readable storage medium may store a computer program or software for execution by processor 12 and/or apparatus 10 to perform any of the methods illustrated in FIGS. 1-4.

Additionally or alternatively, in some embodiments, apparatus 10 may include an input and/or output device (I/O device). In certain embodiments, apparatus 10 may further include a user interface, such as a graphical user interface or touchscreen.

In certain embodiments, memory 14 stores software modules that provide functionality when executed by processor 12. The modules may include, for example, an operating system that provides operating system functionality for apparatus 10. The memory may also store one or more functional modules, such as an application or program, to provide additional functionality for apparatus 10. The components of apparatus 10 may be implemented in hardware, or as any suitable combination of hardware and software. According to certain example embodiments, processor 12 and memory 14 may be included in or may form a part of processing circuitry or control circuitry.

As used herein, the term “circuitry” may refer to hardware-only circuitry implementations (e.g., analog and/or digital circuitry), combinations of hardware circuits and software, combinations of analog and/or digital hardware circuits with software/firmware, any portions of hardware processor(s) with software (including digital signal processors) that work together to cause an apparatus (e.g., apparatus 10) to perform various functions, and/or hardware circuit(s) and/or processor(s), or portions thereof, that use software for operation but where the software may not be present when it is not needed for operation. As a further example, as used herein, the term “circuitry” may also cover an implementation of merely a hardware circuit or processor (or multiple processors), or portion of a hardware circuit or processor, and its accompanying software and/or firmware.

According to certain embodiments, apparatus 10 may be controlled by memory 14 and processor 12 to obtain an RNA sequence from a protein data bank comprising a plurality of RNA sequences. Apparatus 10 may also be controlled by memory 14 and processor 12 to generate a plurality of candidate RNA secondary structures by passing the RNA sequence through at least one RNA secondary structure prediction model. Apparatus 10 may further be controlled by memory 14 and processor 12 to assemble via a RNA tertiary structure generator, the plurality of candidate RNA secondary structures into a plurality of RNA tertiary structures. In addition, apparatus 10 may be controlled by memory 14 and processor 12 to rank the plurality of RNA tertiary structures by implementing a plurality of thermodynamic molecular machine learning models. Further, apparatus 10 may be controlled by memory 14 and processor 12 to determine a structural binding affinity of small-molecule based on the ranked plurality of RNA tertiary structures.

In some example embodiments, an apparatus (e.g., apparatus 10 and/or apparatus 20) may include means for performing a method, a process, or any of the variants discussed herein. Examples of the means may include one or more processors, memory, controllers, transmitters, receivers, and/or computer program code for causing the performance of the operations.

Certain example embodiments may be directed to an apparatus that includes means for obtaining an RNA sequence from a protein data bank comprising a plurality of RNA sequences. The apparatus may also include means for generating a plurality of candidate RNA secondary structures by passing the RNA sequence through at least one RNA secondary structure prediction model. The apparatus may further include means for assembling, via a RNA tertiary structure generator, the plurality of candidate RNA secondary structures into a plurality of RNA tertiary structures. In addition, the apparatus may include means for ranking the plurality of RNA tertiary structures by implementing a plurality of thermodynamic molecular machine learning models. Further, the apparatus may include means for determining a structural binding affinity of small-molecule based on the ranked plurality of RNA tertiary structures.

Certain embodiments described herein provide several technical improvements, enhancements, and/or advantages. In some embodiments, it may be possible to provide an AI approach that does not require any pretraining or existing RNA structures. According to other embodiments, it may be possible to generate a collection of possible competing RNA tertiary structures that may exist at physiological temperatures, along with a thermodynamic ranking that accounts for energetic and entropic contributions to each structure's free energy.

In other embodiments, it may be possible to predict RNA tertiary structures that can be applied to drug discovery and biotechnology. For instance, it may be possible to utilize the predicted RNA tertiary structures to determine small molecules that bind to specific RNA structures for disease treatment, and utilizes such structures in structure-based drug design. In other embodiments, it may be possible to provide a dynamic RNA tertiary structure prediction model that accounts for different environmental conditions such as, for example, temperature.

In further embodiments, it may be possible to provide neural network models that are small/simple enough to be trained on a CPU rather than a GPU. Additionally, the approach of certain embodiments do not require co-evolutionary information to predict RNA structures (extension of not requiring 3D structures). Other embodiments may provide an integration with experimental data (chemical probing and nuclear magnetic resonance (NMR)). According to other embodiments, it may be possible to provide a hierarchical modeling of RNA structures (e.g., the low-resolution to high-resolution score functions described herein).

A computer program product may include one or more computer-executable components which, when the program is run, are configured to carry out some example embodiments. The one or more computer-executable components may be at least one software code or portions of it. Modifications and configurations required for implementing functionality of certain example embodiments may be performed as routine(s), which may be implemented as added or updated software routine(s). Software routine(s) may be downloaded into the apparatus.

As an example, software or a computer program code or portions of it may be in a source code form, object code form, or in some intermediate form, and it may be stored in some sort of carrier, distribution medium, or computer readable medium, which may be any entity or device capable of carrying the program. Such carriers may include a record medium, computer memory, read-only memory, photoelectrical and/or electrical carrier signal, telecommunications signal, and software distribution package, for example. Depending on the processing power needed, the computer program may be executed in a single electronic digital computer or it may be distributed amongst a number of computers. The computer readable medium or computer readable storage medium may be a non-transitory medium.

In other example embodiments, the functionality may be performed by hardware or circuitry included in an apparatus (e.g., apparatus 10 or apparatus 20), for example through the use of an application specific integrated circuit (ASIC), a programmable gate array (PGA), a field programmable gate array (FPGA), or any other combination of hardware and software. In yet another example embodiment, the functionality may be implemented as a signal, a non-tangible means that can be carried by an electromagnetic signal downloaded from the Internet or other network.

According to an example embodiment, an apparatus, such as a device, or a corresponding component, may be configured as circuitry, a computer or a microprocessor, such as single-chip computer element, or as a chipset, including at least a memory for providing storage capacity used for arithmetic operation and an operation processor for executing the arithmetic operation.

One having ordinary skill in the art will readily understand that the invention as discussed above may be practiced with procedures in a different order, and/or with hardware elements in configurations which are different than those which are disclosed. Therefore, although the invention has been described based upon these example embodiments, it would be apparent to those of skill in the art that certain modifications, variations, and alternative constructions would be apparent, while remaining within the spirit and scope of example embodiments.

### Partial Glossary

EternaFold EF
FARFAR2 Fragment Assembly of RNA with Full-Atom Refinement 2
MD Molecular Dynamics
PK pKiss
RNA Ribonucleic Acid
TM Temperature Map Respiratory Rate
TM-aMD Thermodynamic Map-Accelerated Molecular Dynamics

We claim:

1. A method, comprising:
obtaining a ribonucleic acid (RNA) sequence from a protein data bank comprising a plurality of RNA sequences;
generating a plurality of candidate RNA secondary structures by passing the RNA sequence through at least one RNA secondary structure prediction model;
assembling, via a RNA tertiary structure generator, the plurality of candidate RNA secondary structures into a plurality of RNA tertiary structures;
ranking the plurality of RNA tertiary structures by implementing a plurality of thermodynamic molecular machine learning models; and
determining a structural binding affinity of small-molecule based on the ranked plurality of RNA tertiary structures.

2. The method according to claim 1, further comprising:
filtering the plurality of candidate RNA tertiary structures based on energetic characteristics of the plurality of RNA tertiary structures assembled via the RNA tertiary structure generator; and
filtering the RNA tertiary structures obtained through a molecular dynamics simulation based on a score predicted by a thermodynamic map model.

3. The method according to claim 2, wherein the energetic characteristics comprise at least one of the following:
hydrophobicity,
electrostatics, or
ion addition.

4. The method according to claim 1, wherein the plurality of RNA tertiary structures are assembled based on implementation of Monte Carlo simulations to condition the plurality of RNA tertiary structures.

5. The method according to claim 1, further comprising:
refining the plurality of RNA tertiary structures through simulation under a series of increasingly high-resolution energy functions, each of which models physical interactions in greater detail compared to those prior.

6. The method according to claim 1, further comprising:
simulating the plurality of candidate RNA secondary structures with molecular dynamics.

7. The method according to claim 1, further comprising:
training models based on features of the plurality of RNA tertiary structures; and
extracting a score from the trained models.

8. The method according to claim 1, wherein the ranking of the plurality of RNA tertiary structures is based on temperature and entropy, and external environmental conditions of the RNA sequence.

9. The method according to claim 1, further comprising:
generating a thermodynamic map of the plurality of RNA tertiary structures based on an equilibrium distribution of the plurality of RNA tertiary structures, and the effect of temperature on the equilibrium distribution.

10. An apparatus, comprising:
at least one processor; and
at least one memory storing instructions which, when executed by the at least one processor, cause the apparatus to at least:
obtain a ribonucleic acid (RNA) sequence from a protein data bank comprising a plurality of RNA sequences;
generate a plurality of candidate RNA secondary structures by passing the RNA sequence through at least one RNA secondary structure prediction model;
assemble, via a RNA tertiary structure generator, the plurality of candidate RNA secondary structures into a plurality of RNA tertiary structures;
rank the plurality of RNA tertiary structures by implementing a plurality of thermodynamic molecular machine learning models; and
determine a structural binding affinity of small-molecule based on the ranked plurality of RNA tertiary structures.

11. The apparatus according to claim 10, wherein the instructions, when executed by the at least one processor, further cause the apparatus to:
filter the plurality of candidate RNA tertiary structures based on energetic characteristics of the plurality of RNA tertiary structures assembled via the RNA tertiary structure generator; and
filter the RNA tertiary structures obtained through a molecular dynamics simulation based on a score predicted by a thermodynamic map model.

12. The apparatus according to claim 11, wherein the energetic characteristics comprise at least one of the following:
hydrophobicity,
electrostatics, or
ion addition.

13. The apparatus according to claim 10, wherein the plurality of RNA tertiary structures are assembled based on implementation of Monte Carlo simulations to condition the plurality of RNA tertiary structures.

14. The apparatus according to claim 10, wherein the instructions, when executed by the at least one processor, further cause the apparatus to:
refine the plurality of RNA tertiary structures through simulation under a series of increasingly high-resolution energy functions, each of which models physical interactions in greater detail compared to those prior.

15. The apparatus according to claim 10, wherein the instructions, when executed by the at least one processor, further cause the apparatus to:
simulate the plurality of candidate RNA secondary structures with molecular dynamics.

16. The apparatus according to claim 10, wherein the instructions, when executed by the at least one processor, further cause the apparatus to:
train models based on features of the plurality of RNA tertiary structures; and
extract a score from the trained models.

17. The apparatus according to claim 10, wherein the ranking of the plurality of RNA tertiary structures is based on temperature and entropy, and external environmental conditions of the RNA sequence.

18. The apparatus according to claim 10, wherein the instructions, when executed by the at least one processor, further cause the apparatus to:
generate a thermodynamic map of the plurality of RNA tertiary structures based on an equilibrium distribution of the plurality of RNA tertiary structures, and the effect of temperature on the equilibrium distribution.

19. A non-transitory computer readable medium encoded with instructions that, when executed in hardware, performs a process, the process comprising:
obtaining a ribonucleic acid (RNA) sequence from a protein data bank comprising a plurality of RNA sequences;
generating a plurality of candidate RNA secondary structures by passing the RNA sequence through at least one RNA secondary structure prediction model;
assembling, via a RNA tertiary structure generator, the plurality of candidate RNA secondary structures into a plurality of RNA tertiary structures;
ranking the plurality of RNA tertiary structures by implementing a plurality of thermodynamic molecular machine learning models; and
determining a structural binding affinity of small-molecule based on the ranked plurality of RNA tertiary structures.
